(19) United States
(12) Patent Application Publication
(10) Pub. No.: US 2010/0105742 A1
(43) Pub. Date: Apr. 29, 2010

(54) PEARLESCENT LIQUID COSMETIC COMPOSITION

(75) Inventors: Lei Huang, Trumbull, CT (US); Bijan Harichian, Brookfield, CT (US); Shanling Shi, Southbury, CT (US)

Correspondence Address: UNILEVER PATENT GROUP, 800 SYLVAN AVENUE, AG West S. Wing, ENGLEWOOD CLIFFS, NJ, 07632-3100, US

(73) Assignee: CONOPCO, INC., D/B/A UNILEVER, Englewood Cliffs, NJ (US)

(21) Appl. No.: 12/562,230

(22) Filed: Sep. 18, 2009

Related U.S. Application Data
(63) Continuation-in-part of application No. 12/257,433, filed on Oct. 24, 2008, now abandoned.

(51) Int. Cl. A61K 8/49

(52) U.S. Cl. 514/355

(57) ABSTRACT

A pearlescent liquid cosmetic composition and process for preparation is provided herein. The composition includes 14-25% of a C₁₂-C₂₂ fatty acid; 0.1-1.8% of an alkali metal C₁₂-C₂₂ fatty acid soap by weight of the composition; 0.1-10% niacinamide; and 20-85% water. The lustre effect is achieved by careful control of the level of alkali metal hydroxide used to neutralize the fatty acid.

### CROSS-REFERENCE TO RELATED APPLICATION

This application is a Continuation-In-Part of Ser. No. 12/257,433 filed Oct. 24, 2009, now abandoned.

### BACKGROUND OF THE INVENTION

1. Field of the Invention

The invention concerns a cosmetic composition with pearlescent fatty acid particles and method to generate pearlescence for this composition.

2. The Related Art

Oysters create pearls, a process involving crystallization. A particulate such as sand enters the oyster. Over time the sand becomes coated with alternating layers of calcium carbonate and protein. Carbonate layers are opaque; they reflect a part of the incoming light while transmitting the remainder. The protein layers are transparent. Light passes through the transparent layer whereupon it impinges on a surface of the next calcium carbonate layer. Partial reflectance and transmittance keeps occurring as light moves deeper through the layered pearl. A result of the different internal light reflections is an appearance of a pearl lustre.

Chemists have learned to replicate the phenomena by manipulating pearlizing agents in their cosmetic compositions. Incident light rays travel through a formula in which pearlescent particles are suspended. Light from the particle layers are then reflected back hitting the surfaces of several other particle layers.

U.S. Patent Application Publication 2004/0228888 A1 (Kohlhase et al.) describes a cosmetic composition with up to 12% by weight of C₁₂-C₄₀ fatty acid, a C₁₂-C₄₀ fatty alcohol, an amphiphilic polymer, an associative polymer and/or a siloxane elastomer, sodium and/or potassium hydroxide, and a pigment and/or a dye. Pearlescent emulsions result.

Techniques employing crystallization of fatty acids to achieve pearlescence are adversely quite sensitive to certain types of chemicals. One of these crystallization disrupting chemicals is niacinamide. Unilever markets mostly in Southeast Asia a product known as Fair & Lovely® having levels of fatty acid in excess of 14%, partially neutralized with potassium hydroxide and containing niacinamide as a key active component. This technology is reported in U.S. Pat. No. 4,096,240 (Mathur). Formulas according to this technology are not pearlescent.

Accordingly, it is a focus of the present invention to achieve pearlescent phenomena in high content fatty acid formulas, especially those requiring components such as niacinamide which may inhibit the lustre effect.

### SUMMARY OF THE INVENTION

A pearlescent liquid cosmetic composition is provided which includes:
from 14 to 25% of a C₁₂-C₂₂ fatty acid by weight of the composition;
from 0.1 to 1.8% of an alkali metal C₁₂-C₂₂ fatty acid soap by weight of the composition;
from 0.1 to 10% of niacinamide by weight of the composition; and
from 20 to 85% of water by weight of the composition.

Further, a method is provided for achieving pearlescence in a liquid cosmetic composition that includes niacinamide, the method including:
charging a vessel with a mixture of C₁₂-C₂₂ fatty acid and water;
adding an aqueous alkali metal hydroxide in portions to the mixture in an amount equivalent to neutralize from 5 to 8% by weight of the fatty acid in the mixture over a first period of time;
optionally acidifying the mixture of step (B) with a strong Lowry-Bronsted acid to neutralize the alkali metal hydroxide down to a level equivalent to neutralize between 0 and 3% by weight of the fatty acid over a second period of time;
optionally adding an aqueous alkali metal hydroxide in portions to the mixture of step (C) in an amount equivalent to raise the hydroxide level to neutralize from 5 to 8% by weight of the fatty acid in the mixture over a third period of time;
charging niacinamide to the vessel in one or more of steps (A), (B), (C) or (D);
attaining in the vessel a resultant mixture having a composition including:
from 14 to 25% of the C₁₂-C₂₂ fatty acid by weight of the composition;
from 0.1 to 1.8% of an alkali metal C₁₂-C₂₂ fatty acid soap by weight of the composition;
from 0.1 to 10% of niacinamide by weight of the composition; and
from 20 to 85% of water by weight of the composition;
removing from the vessel the resultant mixture as a pearlescent liquid cosmetic composition.

### DETAILED DESCRIPTION OF THE INVENTION

Now it has been discovered that a pearlescent lustre can be imparted to a high fatty acid content formula even in the presence of niacinamide. The lustre effect is achieved by careful control of the level of alkali metal hydroxide neutralized fatty acid, i.e. alkali metal soap. Levels of the soap must be no higher than 1.8% by weight of the composition.

Accordingly, a first important component of the present invention is that of a C₁₂-C₂₂ fatty acid. Typical fatty acids include those of lauric, myristic, palmitic, stearic, isostearic, oleic, linoleic, linolenic, behenic and acid mixtures thereof. Most preferred is stearic acid.

Amounts of the fatty acid may range from 14 to 25%, preferably from 15 to 22%, optimally from 16 to 20% by weight of the composition.

A second component of the present invention will be that of an alkali metal C₁₂-C₂₂ fatty acid soap. The soap will be created in situ by neutralization of a portion of the fatty acid with an alkali metal basic substance such as potassium hydroxide and/or sodium hydroxide. Potassium hydroxide is preferred. Typical soaps include but are not limited to potassium stearate, sodium stearate, potassium isostearate, sodium isostearate, potassium palmitate, sodium palmitate, potassium oleate, sodium oleate, potassium myristate, sodium myristate, potassium linoleate, sodium linoleate, potassium linolenate, sodium linolenate, potassium behenate, sodium behenate and soap mixtures thereof. Most preferred are potassium stearate and sodium stearate.

Amounts of the alkali metal C₁₂-C₂₂ fatty acid soap may range from 0.1 to 1.8%, preferably from 0.5 to 1.4%, optimally from 0.8 to 1.2% by weight of the composition.

A further component of compositions according to the present invention is niacinamide. An alternative name for niacinamide is Vitamin B3. Amounts of niacinamide may range from 0.1 to 10%, preferably from 0.5 to 5%, optimally from 1 to 3% by weight of the composition.

A desirable but optional feature of processing components to achieve a pearlescent lustre is the use of a strong mineral acid to cyclically adjust pH. The term “strong mineral acid” is one in accord with the Lowry-Bronsted theory. Typical strong acids include hydrochloric, sulfuric, nitric and phosphoric acids. Most preferred is hydrochloric acid.

Resultant from the cyclic neutralization in situ will be an alkali metal salt of a strong Lowry-Bronsted acid. Typical salts include sodium chloride, potassium chloride, sodium sulfate, potassium sulfate, sodium nitrate, potassium nitrate, sodium phosphate and potassium phosphate. Amounts of the alkali metal salt of the strong Lowry-Bronsted acid may range from 0.05 to 0.5, preferably from 0.05 to 0.3, optimally from 0.05 to 0.2% by weight of the composition.

Advantageously, the C₁₂-C₂₂ fatty acid as a result of the process may crystallize with an average particle size ranging from 10,000 to 150,000, preferably from 30,000 to 120,000, and optimally from 40,000 to 80,000 nm to provide a pearlescent lustre to the composition.

The pearlescent cream and/or lotion cosmetic compositions of this invention will be oil and water emulsions. These emulsions generally will have a continuous water phase and a discontinuous oil phase, i.e. they will be oil-in-water emulsions. In some embodiments, compositions of this invention will be oil continuous forming a water-in-oil emulsion. Other embodiments may be triplex emulsions being of the oil-in-water-in-oil or the water-in-oil-in-water variety. For all the embodiments, the amount of water present may range from 20 to 85%, preferably from 50 to 80%, optimally from 70 to 80% by weight of the composition.

Compositions of the present invention may have a viscosity which can range from about 40 Pascal-second (PaS) at a shear rate of 1 reciprocal second (1/s), preferably in a viscosity ranging from about 40 PaS to about 300 PaS, more preferably from about 40 to about 100 PaS. Viscosity is measured using any viscometer or Rheometer with a shear rate of 1/s, at ambient temperature 25° C. Suitable viscometers/rheometers are Brookfield, Haake and Bohlin with cone and plate fixtures.

A useful component of compositions according to the present invention is that of an emollient, especially a liquid oil emollient. Amounts of the emollient may range from about 0.1 to about 40%, preferably from about 1 to about 30%, optimally from about 5 to about 25% by weight of each of the first and second compositions.

Emollients may be selected from hydrocarbons, esters, C₈-C₆₀ alcohols, ethers, silicones and combinations thereof. Hydrocarbons suitable for the invention include petrolatum, mineral oil, isoparaffins, polyalpha-olefins and polybutenes. Liquid oil emollients are preferred. By term “liquid” is meant a material pourable at 25° C.

Suitable esters include both natural and synthetic materials. Among the natural esters are triglyceride oils such as castor oil, safflower oil, canola oil, cottonseed oil, corn oil, olive oil, sunflower seed oil, almond oil, avocado oil, palm oil, sesame oil, squalene and maleated soybean oil. Mono- and di-glycerides may also be employed. Illustrative are glycerol monostearate, glycerol di-stearate, glycerol monopalmitate, glycerol monolaurate, glycerol di-laurate, glycerol di-palmitate, glycerol mono-oleate and combinations thereof.

Synthetic esters are represented by C₁-C₂₄ alkyl and alkenyl esters of C₁₀-C₂₄ fatty acids.

Examples include hexyl laurate, isohexyl laurate, isohexyl palmitate, isopropyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, lauryl lactate, myristyl lactate, cetyl lactate, oleyl myristate, oleyl stearate, oleyl oleate, isopropyl myristate, isostearyl palmitate, tridecyl salicylate, C₁₂-C₁₅ alkyl benzoate and combinations thereof.

Suitable fatty alcohols may be those chosen from are lauryl, myristyl, cetyl, hexadecyl, stearyl, isostearyl, hydroxystearyl, oleyl, ricinoleyl, behenyl, erucyl, 2-octyl dodecanyl alcohols and mixtures thereof.

Emollients may also include fatty alcohol ethers. These may include ethoxylated or non-ethoxylated fatty alcohols of 10 to 24 carbon atoms including the lauryl, cetyl, stearyl, isostearyl, oleyl and cholesterol alcohols. When ethoxylated the alcohols have attached thereto from 1 to 50 ethylene oxide groups and/or 1 to 50 propylene oxide groups.

Silicone oils may be useful emollients. These can include dimethyl polysiloxane, methyl phenyl polysiloxane and alkoxylated siloxanes (dimethicone copolyols). Particularly preferred are cyclomethicone, dimethicone and combinations thereof.

Sunscreen agents may be incorporated into the compositions. The term “sunscreen” is used to denote ultraviolet ray-blocking compounds inhibiting absorption within the wavelength region between 290 and 420 nm. These compounds may either be organic or inorganic. The organic compounds are preferred. When the sunscreen is inorganic and serves as the sole sun protective substance, it should be present at levels ranging from about 5 to 30%, preferably from about 8 to 15% by weight.

Typical inorganic sunscreens include titanium dioxide, zinc oxide, iron oxide and combinations thereof. Most preferred is titanium dioxide, especially having an average particle size no higher than 700 nm, preferably no higher than 200 nm, optimally less than 35 nm.

Organic sunscreens may be classified into five groups based upon their chemical structures; para-amino benzoates; salicylates; cinnamates; benzophenones; coumarins; azoles and miscellaneous chemicals including menthyl anthralinate. Also polymeric particles may be useful such as polyethylene and polyamides. Organic sunscreen compound will range in amount anywhere from about 0.1 to about 25%, optimally from about 1 to about 15%, more preferably from about 5 to about 10% by weight of the composition. Preferred sunscreen compounds are octyl methoxy cinnamate and Avobenzone, commercially available respectively as Parsol MCX® and Parsol 1789®.

Humectants of the polyhydric alcohol-type may also be included in the compositions of this invention. The humectant aids in increasing the effectiveness of the emollient, reduces scaling, stimulates removal of built-up scale and improves skin feel. Typical polyhydric alcohols include polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,2-butylene glycol, 1,2,6-hexanetriol, ethoxylated glycerol, propoxylated glycerol and mixtures thereof. For best results the humectant is preferably glycerol known also as glycerin. The amount of humectant may range anywhere from about 0.1 to about 40%, preferably between about 1 and about 15% by weight of the composition.

Compositions of the invention can also include thickeners/viscosifiers in amounts from about 0.01 to about 10% by weight of the composition. As known to those skilled in the art, the precise amount of thickeners can vary depending upon the consistency and thickness of the composition which is desired. Exemplary thickeners are magnesium aluminum silicate (Veegum®), guar gums (such as Jaguar HP-120®), xanthan gum, sodium carboxymethyl cellulose, hydroxyalkyl and alkyl celluloses, and crosslinked acrylic acid polymers such as those sold by Lubrizol under the Carbopol® trademark.

Preservatives can desirably be incorporated into both the first and second compositions of this invention to protect against the growth of potentially harmful microorganisms. While it is in the aqueous phase that microorganisms tend to grow, microorganisms can also reside in the oil phase. As such, preservatives which have solubility in both water and oil are preferably employed in the present compositions. Suitable traditional preservatives for compositions of this invention are alkyl esters of para-hydroxybenzoic acid. Other preservatives which have more recently come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preferred preservatives are methyl paraben, imidazolidinyl urea, sodium dehydroxyacetate, propyl paraben and benzyl alcohol. The preservatives should be selected having regard for the use of the composition and possible incompatibilities between the preservatives and other ingredients in the emulsion. Preservatives are preferably employed in amounts ranging from about 0.01% to about 2% by weight of the composition.

Emulsifiers can be included in the compositions. These materials may be selected from nonionic, anionic, cationic or amphoteric emulsifying agents. Amounts of these materials may range from about 0.1 to about 10%, preferably from about 1 to about 5% by weight. Illustrative of nonionic emulsifiers are alkoxylated compounds based upon C₈-C₂₂ fatty alcohols, C₈-C₂₂ fatty acids and sorbitan.

A variety of skin benefit agents may also be included within the compositions. Illustrative are retinoids, ceramides, phytosphingosines, flavanoids, alpha-hydroxy acids, and combinations thereof. Most preferred are retinoids such as retinol and the C₁-C₂₀ esters of retinol. Amounts of any of these materials may range from 0.00001 to 10%, preferably from 0.01 to 1% by weight of the respective compositions.

Except in the operating and comparative examples, or where otherwise explicitly indicated, all numbers in this description indicating amounts of material ought to be understood as modified by the word “about”.

The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.

The following examples will more fully illustrate the embodiments of this invention. All parts, percentages and proportions referred to herein and in the appended claims are by weight unless otherwise illustrated.

### Example 1

Compositions of this invention may be prepared in a process which is herein described. Components of phases A and B are reported in Table I below.

TABLE I

| | |
|---|---|
| CHEMICAL NAME | WEIGHT % |

Phase A

| | |
|---|---|
| Stearic Acid | 17.90 |
| Cetyl Alcohol | 0.50 |
| Dimethicone | 1.00 |
| Parsol MCX ® | 2.00 |
| Niacinamide | 1.00 |

Phase B

| | |
|---|---|
| DMDM Hydantoin ® (55% Active in Water) | 0.20 |
| Glycerin | 1.00 |
| Potassium Hydroxide (45% Active in Water) | * |
| Water | Balance |

* Adjustable amounts

Phase A containing stearic acid and the other organic components is heated to 80° C. Phase B formulated with an amount of potassium hydroxide calculated to neutralize 7% of the stearic acid is heated to 80° C. and mixed thoroughly. Phase A is then slowly added to phase B maintaining the 80° C. temperature with slow mixing and homogenization. The combined batch is then cooled to 60° C. over a 15 minute period. Thereafter the batch temperature is increased to 65-70° C. within 15 minutes. Hydrochloric acid is dropped into the batch to neutralize potassium hydroxide so that a 3% amount of the stearic acid could possibly be neutralized by the alkali. The batch is then cooled to 60° C. over a 15 minute period. Potassium hydroxide is then added to the batch in an amount to neutralize 5% of the stearic acid. This is followed by a cool down of the batch to 50° C. Finally, any other skin benefit ingredients can be added as a Phase C below 50° C. The batch is discharged from the reactor when the temperature in the vessel is below 30° C. Components of the discharged batch will include: 17% stearic acid, 0.9% potassium stearate, and 0.19% potassium chloride.

### Example 2

A series of comparative experiments were conducted to evaluate the effects of different neutralization levels for the stearic acid. Eleven formulas were prepared in accordance with the process (except for the acidification step) outlined under Example 1. Table II lists the formulas, their neutralization levels, fatty acid crystal sizes, stability and pearlescent effect.

TABLE II

| Sample | Neutralization* | Niacinamide | Crystal Size | Emulsion | |
|---|---|---|---|---|---|
| No. | (%) | (%) | (nm) | Stability | Pearlescent Effect |
| 1 | 14 | 0 | 10,000 | Stable+ | Weak (−) |
| 2 | 12 | 0 | 20,000 | Stable+ | Some (+) |
| 3 | 10 | 0 | 30,000 | Stable+ | Perceivable (++) |
| 4 | 10 | 3 | 20,000 | Stable+ | Weak (−) |
| 5 | 7 | 0 | 35,000 | Stable | Perceivable (++) |
| 6** | 5 | 0 | 50,000 | Stable | Very Good (++++) |
| 7 | 5 | 0 | 45,000 | Stable− | Good (+++) |
| 8 | 5 | 3 | 35,000 | Stable | Good (+++) |
| 9 | 5 | 5 | 25,000 | Stable | Perceivable (++) |
| 10 | 3 | 0 | 50,000 | Unstable | Weak (−) |
| 11 | 1 | 0 | 120,000 | Unstable | Very Weak (−) |
| 12 | 0 | 0 | 120,000 | Unstable | Very Weak (−) |

*Based on weight of fatty acid present.
**Neutralization level is fluctuated + and −2%

Sample No. 6 unlike the other Samples 1-5 and 7-12 was prepared by cyclic pH neutralization in accordance with the Example 1 process. Batch size for all of the formulas was 100 gram. Sample No. 6 was prepared in the following manner. Aqueous phase B was heated to 80° C. and mixed well with a propeller mixer spinning at 450 rpm. Phase B contained 3% niacinamide, 1% glycerin, 0.2% DMDM Hydantoin (55% active), 0.55% potassium hydroxide (45% active equivalent to neutralize 7% stearic acid), and 73.85% water. Oily phase A was heated to 80° C. Phase A contained 17.9% stearic acid, 2% Parsol MCX®, 1% dimethicone and 0.5% cetyl alcohol. Slowly phase A was added to phase B at 80° C., with continuous mixing and homogenization.

The combined phases were then cooled to 60° C. within 15 minutes. At this point the batch viscosity became higher. Temperature was then increased to 65-70° C. over a 15 minute period. Thereafter the batch viscosity became thinner. Then 0.69 gram (4 M) hydrochloric acid was added to neutralize the potassium hydroxide to an extent that only 3% stearic acid could be theoretically neutralized by the hydroxide. The batch was then cooled to 60° C. over a further 15 minute interval. Next 0.157 gram potassium hydroxide (45% active) was added to adjust neutralization levels to 5% of the stearic acid. Mixing was continued and the batch cooled below 30° C. Crystal size, emulsion stability and pearlescent effect were then determined on the resultant composition.

Table II reveals that as the level of neutralization decreases from 14%, the crystal size and pearlescent effect increase. For instance, Sample No. 1 has crystal size of 10,000 nm and a weak pearlescent effect. By contrast, Sample No. 3 at 10% neutralization triples the crystal size to 30,000 nm and results in an already perceivable pearlescent effect. Too little neutralization is undesirable. For instance, Sample No. 10 with 3% neutralization albeit having a crystal size of 50,000 nm, provided an unstable emulsion and a weak pearlescent effect. Sample No. 9 with 5% niacinamide gave a crystal size of 25,000 nm but a just perceivable pearlescent effect. By contrast, Sample No. 7 having an identical 5% neutralization level as Sample No. 9 but without niacinamide resulted in a much larger crystal size of 45,000 nm and a good pearlescent effect.

Sample No. 6 which cycled the pH with an acidification step (HCl) resulted in a crystal size of 50,000 nm and a very good pearlescent effect. This contrasts to Sample No. 7 without pH cycling and at the same 5% overall neutralization level gave a slightly smaller crystal size of 45,000 nm and slightly smaller pearlescent effect.

### Example 3

Another representative embodiment of the present invention is prepared in a fashion similar to that described in Example 1. The resultant formula has the composition outlined under Table III.

TABLE III

| CHEMICAL NAME | WEIGHT % |
|---|---|
| Stearic Acid | 17.90 |
| Glycerin | 1.00 |
| Niacinamide | 1.00 |
| Isopropyl Myristate | 0.75 |
| Octylmethoxycinnamate | 0.75 |
| Cetyl Alcohol | 0.53 |
| Dimethicone | 0.50 |
| Titanium Dioxide (in 40% Isopropylmyristate) | 0.50 |
| Butylmethoxydibenzoylmethane | 0.40 |
| 2-Phenoxyethanol | 0.40 |
| Methyl p-hydroxybenzoate | 0.20 |
| Potassium Stearate | 0.15 |
| Potassium Chloride | 0.15 |
| Propyl-p-hydroxybenzoate | 0.10 |
| Disodium EDTA | 0.04 |
| DL-alpha-Tocopheryl Acetate | 0.01 |
| Water | balance |

What is claimed is:

1. A pearlescent liquid cosmetic composition comprising:
(i) from 14 to 25% of a C₁₂-C₂₂ fatty acid by weight of the composition;
(ii) from 0.1 to 1.8% of an alkali metal C₁₂-C₂₂ fatty acid soap by weight of the composition;
(iii) from 0.1 to 10% of niacinamide by weight of the composition; and
(iv) from 20 to 85% of water by weight of the composition.

2. The composition according to claim 1 further comprising from 0.05 to 0.5% of an alkali metal salt of a strong Lowry-Bronsted acid by weight of the composition.

3. The composition according to claim 2 wherein the alkali metal salt of a strong Lowry-Bronsted acid is selected from the group consisting of sodium chloride, potassium chloride, sodium sulfate, potassium sulfate, sodium nitrate, potassium nitrate, sodium phosphate, potassium phosphate and mixtures thereof.

4. The composition according to claim 1 wherein the C₁₂-C₂₂ fatty acid is stearic acid and the soap is sodium stearate or potassium stearate.

5. The composition according to claim 1 wherein the C₁₂-C₂₂ fatty acid is present from 16 to 20% by weight.

6. The composition according to claim 1 wherein the alkali metal C₁₂-C₂₂ fatty acid soap is present from 0.5 to 1.2% by weight of the composition.

7. The composition according to claim 1 wherein the C₁₂-C₂₂ fatty acid is in crystallized form having an average particle size ranging from 20,000 to 150,000 nm to provide a pearlescent lustre to the composition.

8. A method for imparting pearlescence to a liquid cosmetic composition comprising niacinamide, the method comprising:
(A) charging a vessel with a mixture of C₁₂-C₂₂ fatty acid and water;
(B) adding an aqueous alkali metal hydroxide in portions to the mixture in an amount equivalent to neutralize from 5 to 8% by weight of the fatty acid in the mixture over a first period of time;
(C) optionally acidifying the mixture of step (B) with a strong Lowry-Bronsted acid to neutralize the alkali metal hydroxide down to a level equivalent to neutralize between 0 and 3% by weight of the fatty acid over a second period of time;
(D) optionally adding an aqueous alkali metal hydroxide in portions to the mixture of step (C) in an amount equivalent to raise the hydroxide level to neutralize from 5 to 8% by weight of the fatty acid in the mixture over a third period of time;
(E) charging niacinamide to the vessel in one or more of steps (A), (B), (C) or (D);
(F) attaining in the vessel a resultant mixture having a composition comprising:
(i) from 14 to 25% of the C₁₂-C₂₂ fatty acid by weight of the composition;
(ii) from 0.1 to 1.8% of an alkali metal C₁₂-C₂₂ fatty acid soap by weight of the composition;
(iii) from 0.1 to 10% of niacinamide by weight of the composition; and
(iv) from 20 to 85% of water by weight of the composition;
(G) removing from the vessel the resultant mixture as a pearlescent liquid cosmetic composition.

9. The method according to claim 8 wherein the composition further comprises from 0.05 to 0.5% of an alkali metal salt of a strong Lowry-Bronsted acid by weight of the composition.

10. The method according to claim 9 wherein the alkali metal salt of a strong Lowry-Bronsted acid is selected from the group consisting of sodium chloride, potassium chloride, sodium sulfate, potassium sulfate, sodium nitrate, potassium nitrate, sodium phosphate, potassium phosphate and mixtures thereof.
